(19) United States
(12) Patent Application Publication
(10) Pub. No.: US 2016/0266094 A1
(43) Pub. Date: Sep. 15, 2016

(54) CELLULAR BARCODE

(71) Applicant: University of Iowa Research Foundation, Iowa City, IA (US)

(72) Inventors: James A. Ankrum, Iowa City, IA (US); Anthony J. Burand, JR., Iowa City, IA (US)

(21) Appl. No.: 15/068,106

(22) Filed: Mar. 11, 2016

Related U.S. Application Data
(60) Provisional application No. 62/133,026, filed on Mar. 13, 2015.

(51) Int. Cl. G01N 33/50; G01N 21/64; G01N 33/58

(52) U.S. Cl. CPC G01N 33/5005; G01N 33/588; G01N 33/582; G01N 21/6428; G01N 2021/6439

(57) ABSTRACT

The present invention provides novel barcode structures and compositions and methods of making and using such barcodes, in particular for cellular labeling.

### CLAIM OF PRIORITY

This application claims the benefit of priority of U.S. Provisional Patent Application No. 62/133,026, filed on 13 Mar. 2015, the benefit of priority of which is claimed hereby, and which is incorporated by reference herein in its entirety.

### BACKGROUND

The role of single cells in physiological and pathological environments has become critical to the study of stem cell niches, tumor biology, and regenerative medicine. While traditional biological tools provide a picture of biological processes, the picture is actually an average of the cell population and does not represent the diversity of phenotypes present in that population as multiple cells are pooled together and analyzed as a single data point. Single cell analysis platforms, including single-cell PCR, flow cytometry, and single-cell micro-wells have begun to reveal the true diversity that is present in biology. However, the ability to fully characterize single cells across multiple platforms and in multicellular environments is limited by the inability to uniquely identify and track single cells.

### SUMMARY OF THE INVENTION

The present invention is comprised of a set of fluorescent beads that serve as single cell barcodes and a method of labeling and tracking cells over time. For example, the invention allows for the unique identification of single cells across time and location in a multicellular environment.

One embodiment provides a barcode comprising a particle core of about 200 nm to 2000 nm in size and at least one fluorescent light emitting entity associated with said particle core. In one embodiment, the core comprises a metal, a polymer or a combination thereof. In another embodiment, the metal is gold, silver or a combination thereof. In one embodiment, the polymer is PLGA, PLA, PGA, polystyrene, silicon, PCL or a combination thereof.

One embodiment, the particle core is surface modified. In one embodiment, the surface is modified so as to have biotin, streptavidin, an amino group, a carboxyl group, an antibody or combination thereof on the surface of the particle core. In one embodiment, the at least one fluorescent entity is an organic fluorophore, a synthetic fluorophore, fluorescent nanocrystal, a polymeric fluorescing molecule or a combination thereof. In one embodiment, the fluorescence entity is fluorescein, FITC, texas red, Alexa Fluor dyes, cyanine dyes or a combination thereof. In another embodiment, the fluorescent nanocrystal is a quantum dot (QDot). In another embodiment, the polymeric fluorescing molecule is a brilliant violet dye, a long-chain dialalkylcarbocyanine dye, or a combination thereof. In another embodiment, the at least one fluorescent entity is directly or indirectly attached to the core. In another embodiment, the at least one fluorescent entity is attached to the core by an antibody, a biotin/streptavidin attachment, electrostatic interactions or a combination thereof. In one embodiment, multiple antibodies with fluorophores are stacked on the core. In another embodiment, there are sequential biotin-streptavidin interactions. In one embodiment, there are multiple layers of fluorophores held by electrostatic interactions. In one embodiment, the electrostatic interactions are created by alternating layers of PLL and PAA.

In one embodiment, the barcode further comprises poly-D-lysine.

One embodiment provides a method to label a cell comprising contacting said cell with said barcode described herein. Another embodiment provides a method to identify and/or track cells comprising measuring the fluorescent intensity from the barcode in the labeled cell. In one embodiment, the barcode is read at more than one time point. In another embodiment, the barcode is passed to multiple cell generations. In one embodiment, the barcode is stable for about two or more weeks. In one embodiment, the barcode degrades upon cell death. In another embodiment, the barcode can be detected by flow cytometry, fluorescent microscopy, high content imaging or a combination thereof.

In one embodiment, each barcode is a microparticle 300-900 nm in diameter that has been encoded with a unique combination of three colors of quantum dots (QDots). Barcode particles can be read with fluorescence microscopy and identified by the intensity of the fluorescence emission at three wavelengths (ten intensities of each color produces 1,000 unique codes, 100 intensities produce 1×10⁶). Each cell can be loaded with multiple particles so that on day 0, each cell is associated with multiple identifying codes. As cells divide, barcodes will be distributed to daughter cells. Thus, the lineage of cells can be traced over time until each cell contains only 1 barcode, after which a minimum fraction of the cells will remain labeled for further tracking. Imprinting the entire barcode into each microparticle overcomes limitations of previous strategies that arose due to nanoparticle exocytosis or uptake of nanoparticles after cell death. Particle transfer will also be minimized by using larger particles which are more resistant to exocytosis (8). To prevent barcodes from being transferred to cells when cells undergo apoptosis, a self-destructing mechanism can be included. In one embodiment, QDots will be covalently bonded to a poly-D-lysine (PDL) backbone and then coated, layer-by-layer, onto a particle core (FIG. 1). While poly-D-lysine is resistant to enzymatic degradation and is stable in aqueous environments, it rapidly degrades when cells undergo either apoptosis or necrosis (9). Thus, QDots will remain localized on the particle while the cell is alive, but will become diffuse when the cell dies, destroying the barcode (FIG. 2). Thus, the compositions and methods disclosed herein enable longitudinal tracking of thousands of single cells across multiple generations while also serving as a viability marker. Tracking for days to weeks is desirable as it enables cells to be analyzed in multiple single cell assays and then followed in multicellular environments. Such a capability enables biomarkers that predict future cell function and behaviors.

One embodiment provides a barcode microparticle comprising one or more layers of quantum dot (QDot) labeled poly-D-lysine (PDL) on a core. In one embodiment, the core comprises gold or polystyrene particles. In another embodiment, the gold is colloidal gold particles. In another embodiment, the gold particles are about 250 nm in size. In one embodiment, the polystyrene particles are about 500 nm in size.. In one embodiment, the polystyrene particles are about 900 nm in size. In another embodiment, there are 3 or more layers. One embodiment comprises poly (acylic acid) (PAA), such a negatively charged PAA. In one embodiment the barcode is about 300-500 nm in size. In another embodiment, the barcode is about 500 nm to about 1 μm in size.

One embodiment provides a method for making a barcode described herein comprising, a) providing a core; b) coating the core with a plurality of distinct layers, wherein each layer is applied sequentially, wherein the plurality of layers comprises quantum dot (QDot) labeled poly-D-lysine (PDL), wherein there are at least 3 layers, wherein the QDots are present in each layer at a defined intensity level, wherein each layer comprises a single type of QDot that is different relative to the QDots contained in any other layer. In one embodiment, the core comprises gold or polystyrene particles. In another embodiment, the gold is colloidal gold particles. In one embodiment, the gold particles are about 250 nm in size. In another embodiment, the polystyrene particles are about 500 nm in size. One embodiment further comprises coating each QDot labeled PDL layer with a poly (acylic acid) (PAA), such as negatively charge PAA.

One embodiment provides a method to label a cell comprising contacting said cell with a barcode described herein.

Another embodiment provides a method to identify and/or track cells comprising measuring the fluorescent intensity from the fluorescent entity, such as QDots, of the barcode in the labeled cell. In embodiment, the barcode is read at more than one time point. In another embodiment, the barcode is passed to multiple cell generations. In one embodiment, the barcode is stable for two or more weeks. In another embodiment, the barcode degrades upon cell death.

### BRIEF DESCRIPTION OF THE DRAWINGS

FIGS. 1A-B depict a schematic of fluorescent barcode. A. The reaction of polylysine to fluorophores can be tuned to create unique levels of intensity. (RFI: relative fluorescence intensity) B. Polylysine will be conjugated to QDot 525, QDot 625 or QDot 705 at varying ratios to generate different levels of intensity. The relative intensity of the particle at each wavelength will generate a unique fluorescent signature, or barcode to identify that particle.

FIG. 2 depicts barcodes transferred to progeny and self-destruction. Cells start with multiple barcodes that are then distributed when a cell divides. PDL remains attached to particle core until cell dies, at which time it is degraded due to high levels of reactive oxygen species and the code self-destructs.

FIG. 3 depicts adsorption and emission spectra for QDot 525, QDot 625, and QDot 705. Figure generated by Life Technologies' SpectraViewer.

FIG. 4 depicts generation of a barcode library. The barcode will be built by sequentially layering different colored QDot labeled poly-D-lysine onto the surface of a core particle. The schematic shows an example of 10 different levels of QDot labeling being used for each wavelength. Particles will be divided amongst the 10 levels and labeled. After labeling, particles will be washed to remove unbound PDL, all particles will be mixed, and a poly(acrylic acid) (PAA) layer will be applied. The particles will then be redistributed and the second color of labeled PDL will be adsorbed to the surface of the particles. The process will be repeated until all 3 colored layers have been adsorbed to the surface. To increase code depth, the number of unique labeling levels can be increased to add more diversity within each color.

FIGS. 5A-D depict fluorophore conjugations to polystyrene cores. A) FITC, Alexafluor, Qdot, and Brilliant Violet conjugations to core. B) Dual color Qdot particles. C) Qdot brightness levels via flow cytometry D) Stability of Qdot signal over 3 days.

FIGS. 6A-E depict barcode constructs. A) Direct dye conjugation B) Conjugation to functionalized core C) Streptavidin/avidin/neutravidin-biotin dye conjugation D) Antibody conjugation E) Electrostatic affinity interaction. (i) In some embodiments, molecules attached to the core may have fluorescent entities of the same color. (ii) In some embodiments, molecules attached to the core may have fluorescent entities of two or more colors. (iii) In some embodiments, one color layer may be added to the particle at a time. (iv) In some embodiments, multiple different colors may be added to an additional layer of the particle.

FIG. 7 depicts an example color gradient chamber for three color system. Black dots in chamber represent particles.

### DETAILED DESCRIPTION OF THE INVENTION

The role of single cells in physiological and pathological environments is important to the study of stem cell niches, tumor biology, and regenerative medicine. While traditional biological tools provide a picture of biological processes, the picture is actually an average of the cell population and does not represent the diversity of phenotypes present in that population as multiple cells are pooled together and analyzed as a single data point.

Single cell analysis platforms, including single-cell PCR, flow cytometry, and single-cell micro-wells have begun to reveal the true diversity that is present in biology. However, the ability to fully characterize single cells across multiple platforms and in multicellular environments is limited by the inability to uniquely identify and track single cells.

Herein is provided a solution that is capable of uniquely labeling thousands of single cells across multiple cell generations, is stable for weeks, can be readily repeatedly, and self-destructs when a cell dies. This technology can rely only on routine fluorescence microscopy and is applicable of being use in a diverse array of cell types. This technology enables full single cell characterization of cells alone or in multicellular environments.

Definitions:

In describing and claiming the invention, the following terminology will be used in accordance with the definitions set forth below. Unless defined otherwise, all technical and scientific terms used herein have the same meaning as commonly understood by one of ordinary skill in the art to which this invention belongs. Any methods and materials similar or equivalent to those described herein can be used in the practice or testing of the present invention. Specific and preferred values listed below for radicals, substituents, and ranges are for illustration only; they do not exclude other defined values or other values within defined ranges for the radicals and substituents.

As used herein, the articles “a” and “an” refer to one or to more than one, i.e., to at least one, of the grammatical object of the article. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

The term “isolated” refers to a factor(s), cell or cells which are not associated with one or more factors, cells or one or more cellular components that are associated with the factor(s), cell or cells in vivo.

“Cells” include cells from, or the “subject” is, a vertebrate, such as a mammal, including a human. Mammals include, but are not limited to, humans, farm animals, sport animals and companion animals. Included in the term “animal” is dog, cat, fish, gerbil, guinea pig, hamster, horse, rabbit, swine, mouse, monkey (e.g., ape, gorilla, chimpanzee, or orangutan), rat, sheep, goat, cow and bird.

Quantum dots are semiconductor nanocrystals that emit light when excited. Quantum dots are on the order of a nanometer in size. They are composed of a hundred to a thousand atoms. These semiconductor materials can be made from an element, such as silicon or germanium, or a compound, such as CdS or CdSe. The color of the fluorescence depends not only on the material, but also on the size of the dot. This unique optical property makes quantum dots suitable for labelling cells.

Quantum dot barcodes are unique microscale arrays of quantum dots that can be identified by analyzing the spectrum of light they emit when exposed to a single light source.

The terms “comprises,” “comprising,” and the like can have the meaning ascribed to them in U.S. Patent Law and can mean “includes,” “including” and the like. As used herein, “including” or “includes” or the like means including, without limitation.

Cellular Barcoding

Cellular barcoding enables individual cells to be labeled with unique identifying tags, much like merchandise at the supermarket. Barcodes developed to date have taken a variety of forms that make them applicable for specific applications, but often fall short of wide adoption due to inability to read the code at multiple time points, degradation of the code over time, characteristics that interfere with cell behavior, requirements for specialized equipment, and lack of code depth required to track large populations of cells. To date, the most widely used barcoding technique has been DNA barcoding which relies on unique natural sequences (1) or synthetic sequences (2) integrated into the cell's DNA. DNA barcoding is inheritable and has been used extensively in stem cell biology for lineage tracing. However, DNA barcoding is limited to end-point analysis, as cell lysis and sequencing is required to read the code (1).

Optical barcoding is an alternative strategy that allows serial reading of the code. Several strategies have been developed to create optical barcodes using combinations of fluorescent molecules. Early attempts labeled entire subpopulations of cells with a unique combination of fluorescent dyes (3,4) or induced expression of an artificial cell surface marker (5). These strategies require a priori knowledge of the phenotype of each group of cells. While useful for parallel analysis of different populations of cells via flow cytometry, subpopulation labeling does not allow for single cells to be tracked so changes in phenotype or location can be analyzed over time.

Recently, fluorescent particles have been used to create true single-cell fluorescent barcodes. Castellarnau et al. developed a technique called stochastic particle barcoding (SPB) that constructs a barcode by fixing the location of fluorescent particles in a polymerized gel around each cell (6). Once the cell is encapsulated in the barcoded gel, it can be moved to a new location, its identity read, and the cell can be released through enzymatic degradation of the gel. SPB requires only a standard fluorescent microscope and can uniquely label ˜10,000 cells using a three color system. This strategy has proven useful for analyzing cells in multiple single cell assays while maintaining the identity of each cell. However, encapsulating the cell limits SPB to be used only to identify cells as they are moved from one in vitro assay to another and is not applicable for live cell tracking, lineage tracing, or multicellular applications.

Nanoparticle vesicle encoding (NVE) is a similar strategy that builds a fluorescent code inside each cell instead of around it (7). By sequentially incubating cells with quantum dots (QDots) with different emission spectra, cells randomly accumulate QDot loaded vesicles, with each vesicle containing particles of only one type of QDot. By counting the number of vesicles that contain each type of QDot, a code is generated. Using this sequential incubation approach with three types of QDots, 17,000 unique codes were created (7). NVE created excellent code depth and could be easily read with a conventional fluorescent microscope, however the stability of the code limits this platform to short term analysis (4-12 hours). The number of vesicles containing QDots of each type quickly change as particles are exocytosed and cells undergo routine cell division. In addition, as cells in the population undergo apoptosis, QDots can be internalized by neighboring cells, which alters their barcodes.

Single cell identification and analysis in multicellular environments requires a novel type of barcode. In one embodiment, this code uniquely labels individual cells, simultaneously tracks thousands of cells, is stable for weeks, enables tracking of cell progeny, and self-destructs when the cell dies. In addition, the barcode is compatible with complimentary cell analysis techniques, in particular, reporter systems. The ability to phenotype and then longitudinally track single cells will unlock new avenues of research by enabling detailed studies of cell-cell interactions in multicellular environments and the ability to identify predictors of cell fate.

TABLE 1
Comparison of select barcoding technique to the novel Self-Destructing Barcode

| | | Repeat | | Multigenerational | Multicellular |
|---|---|---|---|---|---|
| Technique | Code Depth | Measurements | Stability | Labeling | Environment |
| DNA Barcode | Increases with | No | Stable for life of cell | Yes | Cells must be |
| | larger code length | | | | lysed to read code |
| Stochastic | ~10⁴ | Yes | Hours, degraded | No | No |
| Particle Barcoding | | | when cell is released | | |
| Nanoparticle | ~10⁴ | Yes | Hours | No | Yes |
| Vesicle Encoding | | | | | |
| Self-Destructing | 10³-10⁶ Codes | Yes | Weeks | Yes | Yes |
| Barcode | | | | | |

Herein is disclosed a novel intracellular barcode that can track thousands of cells for weeks and across multiple generations. The novel cell barcodes described herein are comprised of a particle core with at least one fluorescent light emitting entity associated/attached to the particle core to form a barcode that has a color and brightness signature that can be detected via flow cytometry, conventional fluorescent microscopy, or high content imaging. In one embodiment, the particles can be loaded individually into the cells or multiple particles can be loaded into a cell. These barcodes could be used to track individual cells over time, as well as track progeny as cells divide.

Core

As most materials can be chemically functionalized to allow association with fluorescent entities, the particle core can be composed of a range of metal and polymer materials. Some materials include gold, silver, PLGA, PLA, PGA, polystyrene, silicon, PCL, amongst others. The size of the core can also be modified from about 200 nm to about 2000 nm in size, including about 200 nm, about 300 nm, about 400 nm, about 500 nm, about 600 nm, about 700 nm, about 800 nm, about 900 nm, about 1000 nm, about 1100 nm, about 1200 nm, about 1300 nm, about 1400 nm, about 1500 nm, about 1600 nm, about 1700 nm, about 1800 nm, about 1900 nm, and/or about 2000 nm. Smaller particle cores will have more limited ability to associate with a high number of fluorescent entities, while large particle cores will be able to associate with many fluorescent entities allowing greater differences between fluorescent levels. Smaller particle cores will be more easily internalized by cells, while particles cores larger than 2 microns will have low efficiency of internalization by non-phagocytic cells. In some embodiments the cores may include being magnetic allowing them to be pulled out of solution with the use of a magnet.

The particle core can have a native surface or be surface modified to have biotin, streptavidin, amine groups, carboxyl groups, or antibodies on the surface. In some embodiments the surface modification is achieved through covalent modification, such as EDC/NHS reaction. In some embodiments, the surface modification is achieved through non-covalent modifications such as adsorption of molecules to the particle core surface.

### Fluorescent Entity

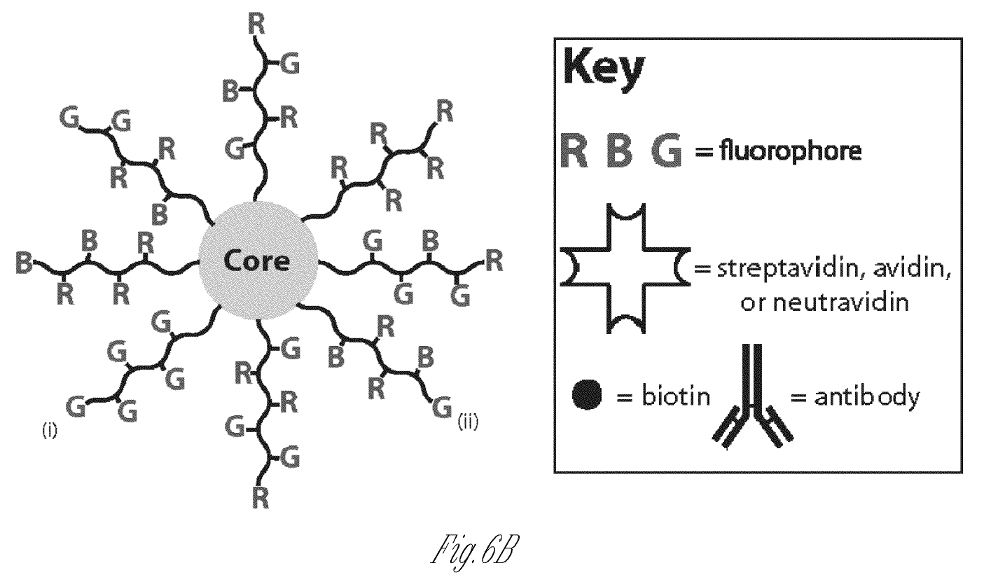

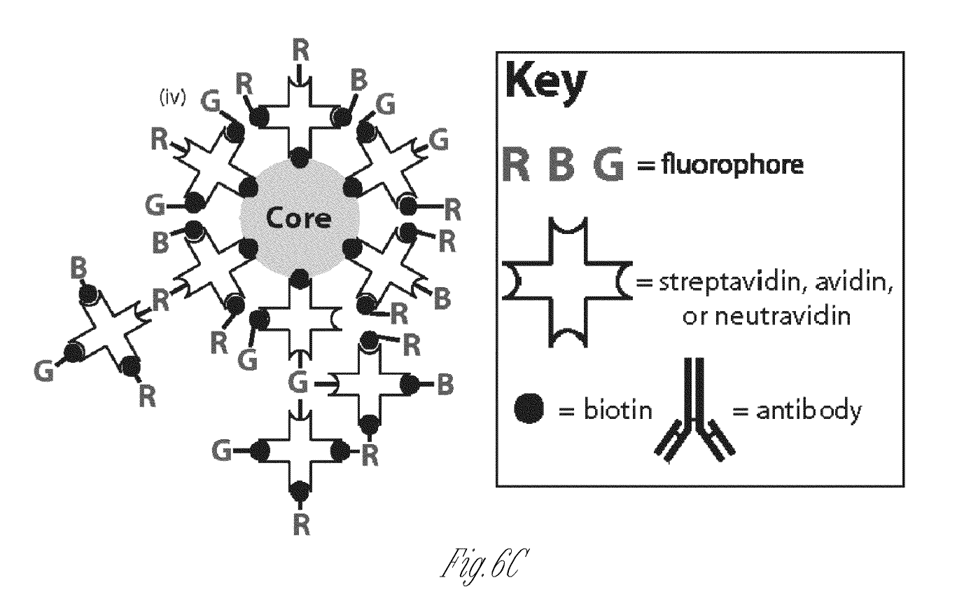

The fluorescent entity attached to the core can include organic or synthetic fluorophores (such as fluorescein, FITC, texas red, Alexa Fluor dyes, cyanine dyes), fluorescent nanocrystals (such as a quantum dot), polymeric fluorescing molecule (such as a brilliant violet dyes, and long-chain dialkylcarbocyanine dyes), or other light emitting molecules attached to the surface (FIG. 5A). Fluorescent molecules may contain/be modified to contain amine, carboxyl, biotin, streptavidin/avidin/neutravidin functionality to aid in attachment to the particle core. In some embodiments, multiple different fluorescent entities may be used on the same particle. In some embodiments one or more different fluorescent colors may be used in combination.

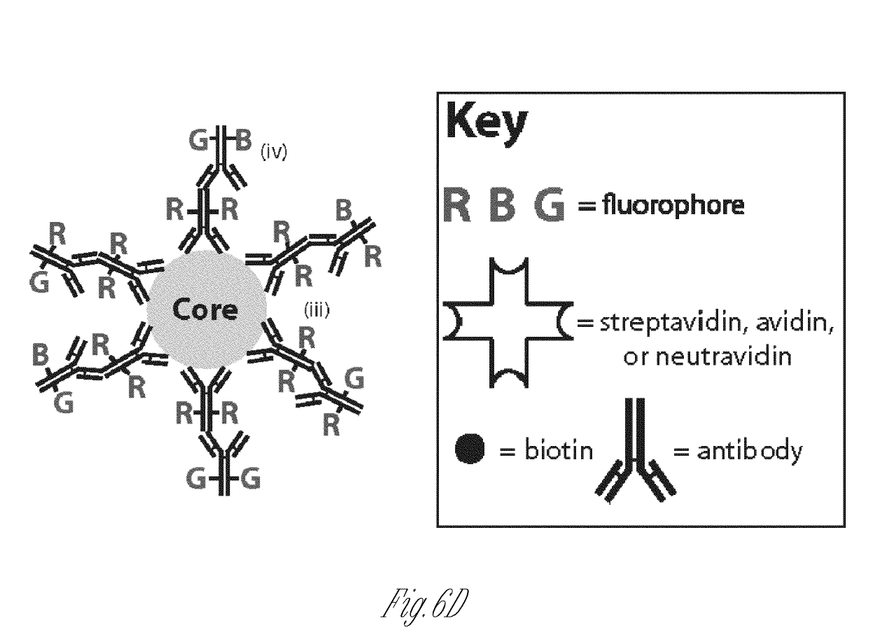

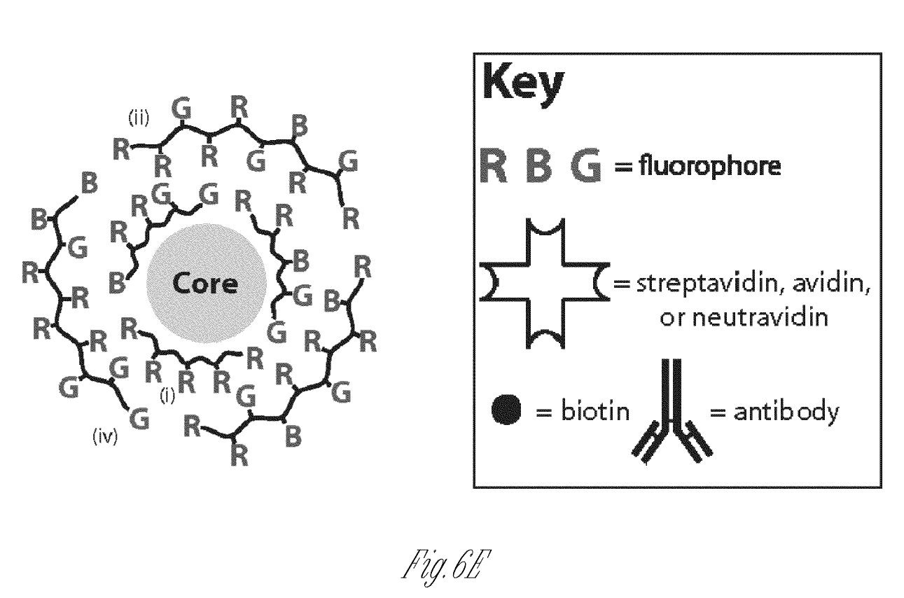

In some embodiments, fluorescent entities may be attached to the core directly through an amine-carboxylic acid conjugation reaction (such as EDC/NHS reaction) (FIG. 6A). In some embodiments, fluorescent entities may be attached to the core indirectly by conjugating the fluorescent entity to a molecule that is itself attached to the particle core (FIG. 6B). In some embodiments, fluorescent entities may be attached to the core via high affinity non-covalent interactions (such as biotin to streptavidin/avidin/neutravidin or antibody: antigen interactions where one binding partner in the high affinity non-covalent interactions is attached to the particle core and the other binding partner is attached to the fluorescent entity (FIG. 6C,D). In some embodiments, fluorescent entities may be attached to the core through electrostatic interactions of a charged core to an oppositely charged fluorescent molecule, such as the interaction between a negatively charged carboxylate core with positively charged polylysine with attached fluorescent entities (FIG. 6E).

To achieve multiple distinct fluorescent codes, different numbers of different kinds of fluorescent entities can be attached to the particle core. By adding fluorescent entities with different emission wavelengths or colors, the code can be changed. In addition, changing the number of fluorescent entities that share a single color that are attached to the particle core will change the brightness of the barcode in a specific channel (FIG. 5C). Variations in fluorescent intensity at a specific wavelength is called a fluorescent level. Varying fluorescent levels can be produced through several different methods. In one method, fluorescent levels can be created through conjugation of different numbers of fluorescent molecules directly to the core or to molecules conjugated to the core (FIG. 6A,B). In another method, fluorescent levels can be created through stacking of multiple antibodies with fluorophores to the core (FIG. 6D). In another method, fluorescent levels can be created though use of sequential biotin-streptavidin interactions (FIG. 6C). In another method, fluorescent levels can be created through multiple layers of fluorophores that are held together through electrostatic interactions (such as alternating PLL and PAA layers) (FIG. 6E).

### Self-Destructing Particles

In some embodiments, particles may be designed to be self-destructing upon cell death. This could be accomplished through layering of the particles with poly-D-lysine which is known to degrade when cells die. Self-destructing mechanisms would prevent the transfer of particles between cells when cells die. Alternatively, only one of the fluorescent colors could be programmed to self-destruct via poly-D-lysine linkage to the particle core. In such an embodiment particles lacking the ‘living’ color would be excluded from analysis while the remaining colors in the barcode could be attached to the particle core through any method of attachment.

### Internalization

Barcode internalization by cells can be facilitated through several methods. In one method barcodes are simply incubated in the media with the cells and cells internalize particles. In another method internalization is facilitated by coating the barcodes in a polycationic molecule such as polylysine, polyarginine, polyethylenimine. In another method internalization is facilitated by conjugation of antibodies to the barcodes that recognize a surface antigen on target cells. In another method, barcodes are introduced to cells using transfection techniques including electroporation, chemical mediated transfection, or mechanical stress mediated transfection (high speed cell deformation).

Each cell can be loaded with one to numerous particles, for example, 10-30 particles, which are distributed to daughter cells upon division and particles can be identified in cells by using, for example, conventional fluorescence microscopy (12).

### Barcode

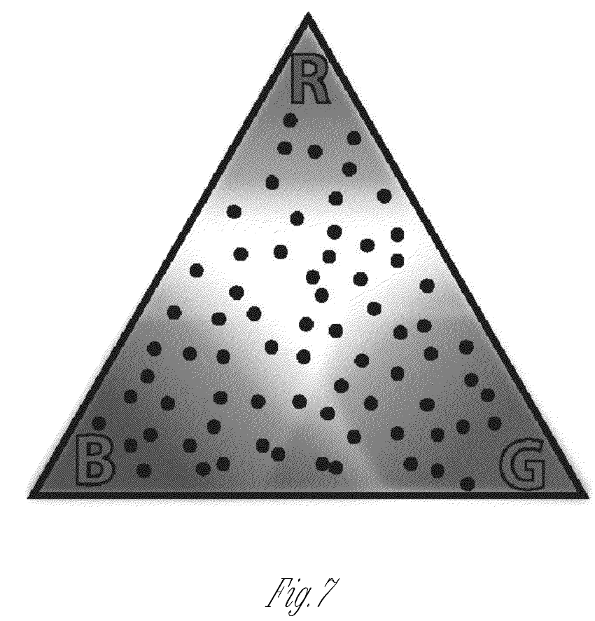

The code of the barcode is defined as the fluorescent level of the attached fluorescent entities at each emission wavelength. Barcodes may be single or multiple colors (FIG. 5A,B). Unique combinations of colors and brightness levels may be produced by concentration gradients in a gradient chamber (FIG. 7). In this gradient chamber, fluorescent entity “sources” or areas of high concentration of the fluorescent entity placed at discrete points around the chamber will allow the fluorescent entities to diffuse creating a concentration gradient of multiple colors. This gradient will allow for unique combinations of fluorescent entities on the particles. Color combinations may be made by directly modulating the amounts of each fluorescent molecule attached to the core or other molecules that are attached to the core (FIG. 6A,B,C,D). Using such a method creates a batch of particles that have the same fluorescent level in at least one emission wavelength. In another method, multiple layers of fluorescent entities on electrostatically charged polymer can also be used (FIG. 6E) to create combinations of fluorescent colors of different fluorescent levels. In some methods, only one color of fluorescent entity is attached to the particle core at a time while in others multiple colors of fluorescent entities are attached simultaneously. In some methods, additional fluorescent entities of a particular color can be added sequentially to modify the level of an existing barcode, or to add an additional fluorescent color to an existing barcode.

### Barcode Identity

Identity of the barcode code can be determined by absolute, maximal, or average values of the fluorescent levels at each fluorescent color, ratios of the color intensities to each other, or ratios of color intensity to a reference color on the particle or a control bead.

The following example is intended to further illustrate certain embodiments of the invention and is not intended to limit the scope of the invention in any way.

### EXAMPLE

### Materials

Poly-D-lysine hydrobromide, 30-70 kDa, Sigma-Aldrich; Poly(Acrylic Acid), sodium salt, 15 kDa, Sigma-Aldrich; Sodium Bicarbonate, Sigma-Aldrich; lonomycin, Sigma-Aldrich; Staurosporine, Sigma-Aldrich; Phosphate Buffered Saline, Sigma Aldrich; MEM-alpha Culture Media, Sigma Aldrich; Trypsin, Sigma Aldrich; Qdot 525, Carboxyl functionalized, Life Technologies; Qdot 625, Carboxyl functionalized, Life Technologies; Qdot 705, Carboxyl functionalized, Life Technologies; Gold Colloid, 250 nm, Fisher Scientific; Polystyrene particles, 500 nm, Carboxyl functionalized, Fisher Scientific; Vivaspin 10 kDa MWCO centrifugal filters, Fisher Scientific; 8 mm biopsy punch, Fisher Scientific; MilliQ Distilled Ultrapure Water; Mesenchymal Stem Cell, RoosterBio; Cell Culture Multiwell Plates, Greiner Bio-One; Assorted Antibodies, R&D Systems; CellStripper non-enzymatic cell dissociation reagent, Corning

### Methods

### Development of Barcode Library

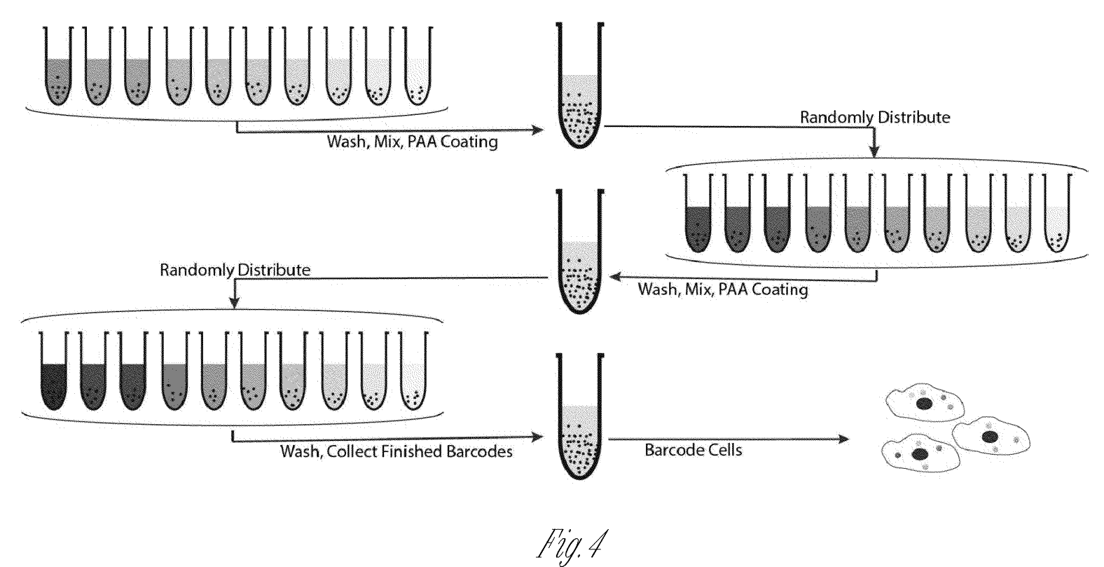

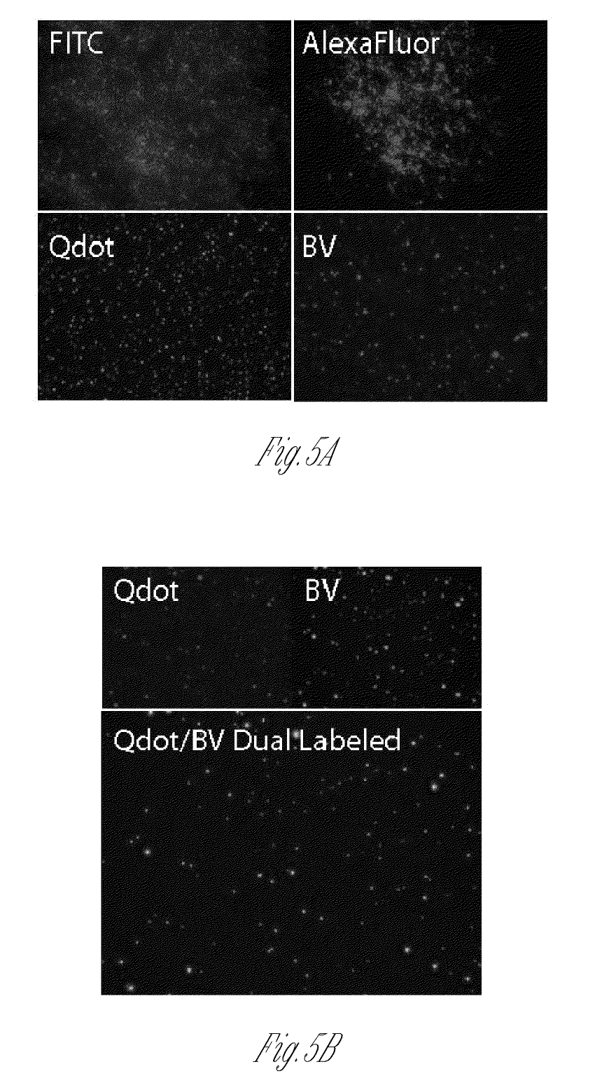

Barcode diversity will be generated through sequential layering of QDot labeled PDL. Each layer of PDL will be conjugated to QDots with non-overlapping emission wavelengths (FIG. 3). By varying the number of QDots conjugated to the PDL, different levels of intensity for each color can be generated. Thus 10 levels of intensity for three colors would produce 103 or 1000 unique barcodes while 25 levels would produce 25³ or 15,625 unique codes.

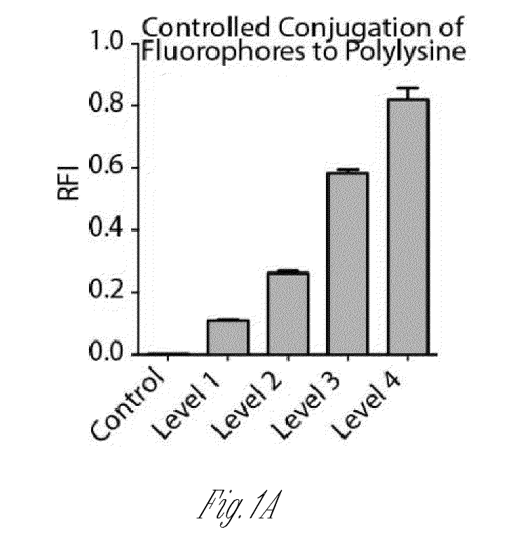

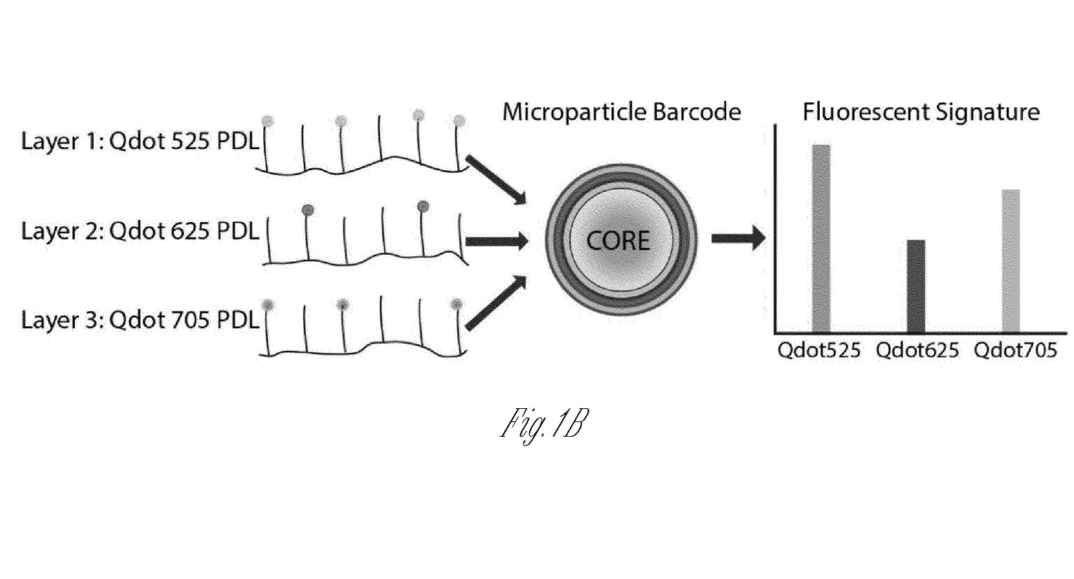

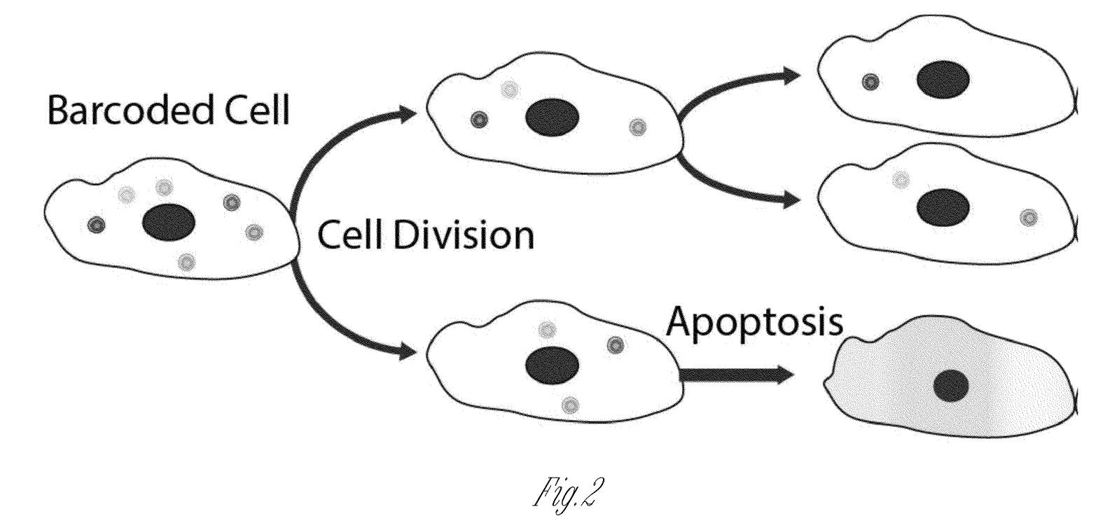

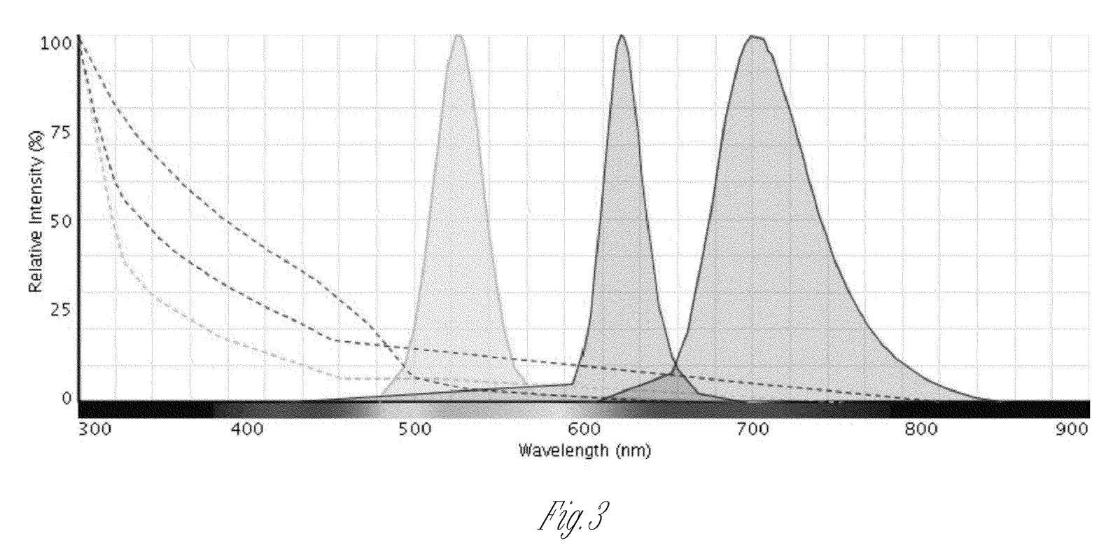

ODot immobilization on PDL: The first step is to generate levels of intensity for each particle by controlling the number of QDots immobilized on each molecule of PDL. The molar ratio of QDots to PDL will be varied and the carboxyl functionalized QDots will be chemically conjugated to PDL (FIG. 1A). Briefly, QDots and PDL will be reacted in a 1 mM solution of NaHCO₃ in the dark at room temperature under constant agitation. Labeled PDL will be separated from unbound QDots using 10 kDa molecular weight cutoff centrifugal membrane filters.

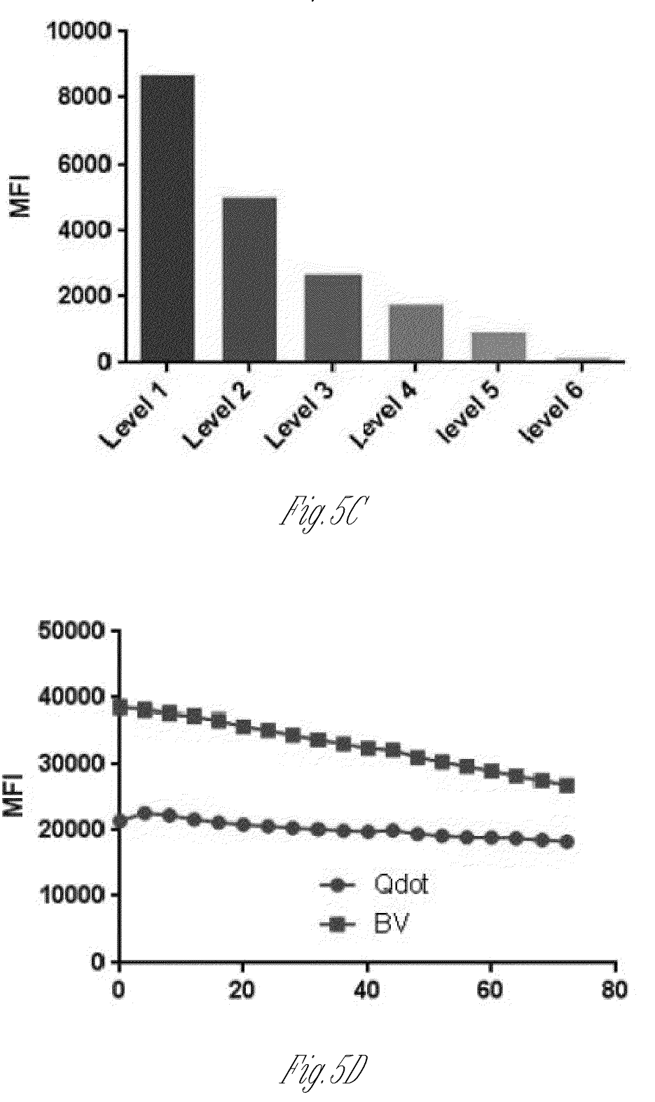

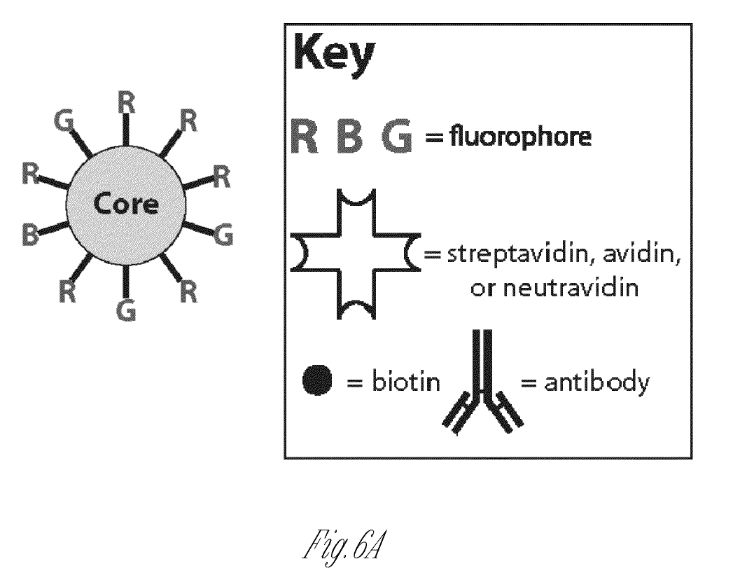

Sequential layering to create code: Barcodes will be constructed on a particle core through layer-by-layer adsorption of labeled PDL to the surface. Two types of particles will be evaluated to serve as the core of the barcode, 250 nm diameter colloidal gold particles and 500 nm polystyrene particles. Larger particles can also be used, as they can be easier to read with routine fluorescence microscopy objectives and filters. Particles will be suspended in a solution of QDot labeled PDL and incubated at RT in the dark for 30 min. Particles will then be centrifuged for 30 min at 16,000 g and washed with ultrapure water. Particles will be washed two more times to remove any unbound PDL. Between each layer of PDL, a layer of negatively charged poly(acrylic acid) (PAA) will be adsorbed to the surface. Particles will be suspended in 20 mM solution of PAA and incubated in the dark with gentle agitation for 30 min. Particles will be washed with ultrapure water to remove unbound PAA. To randomize the combination of emission intensities at each wavelength, particles modified with different levels of PDL of the same color will be thoroughly mixed, and then divided into reaction vessels for the next layer of PDL to be adsorbed. The process will be repeated until all 3 colors of QDot labeled PDL have been adsorbed to the surface and is portrayed schematically in FIG. 4.

Cell Labeling

Cell modification: Human mesenchymal stem cells (MSC) will be used as a model cell to demonstrate the feasibility of tracking single cells through multiple generations. MSCs will be seeded in 6 well plates and grown to a 70% confluence. Barcode particles will be suspended in 1% serum media and then plated on top of the MSC. Cells will be incubated for 4 hours to allow for particle uptake. Cells will then be washed to remove excess barcodes.

Particles per cell: Cells will be imaged at 40× and 63× using a standard inverted fluorescence microscope. Digital image processing will be performed to quantify the number of barcodes in each cell.

Reading Codes/Serial Code Reading: The barcode signature of each particle will be determined through processing of multichannel fluorescence images. As particles are mono-disperse, each is identical in size and amenable to detection through image processing. A region of interest will be placed on the center of each particle and the fluorescence intensity of the barcode at each emission wavelength will be measured. The intensity at each wavelength will then be assigned as the barcode value for that particle. Using live cell imaging, cells will be serially imaged to evaluate particle distribution during mitotic events and the ability to reproducibly identify single cells.

Particle Distribution to Daughter Cells: The ability to track cells over multiple generations depends on efficient distribution of particle to daughter cells during cell division. To identify the limitations of this approach, cells will be modified with barcodes and followed through five population doublings. Cells will be plated modified, and a fraction of the population will be evaluated after each doubling event. Cells will be imaged twice daily to quantity the number of barcodes associated with each cell and the code for each particle will be recorded. As cells reach confluence, cells will be split into larger flasks and serial imaging will continue until the cell population has undergone five population doublings. The specific dynamics of particle distribution will also be evaluated using live cell imaging of cells actively undergoing mitosis.

Particle Transfer: Transfer of barcodes from one cell to another confounds the ability to serially track single cells and should be limited. Based on previous experience with modifying cells with large 500 nm to 1 μm diameter particles it is believed that larger particles will minimize particle transfer. To test this, 250 nm gold core and 500 nm polystyrene core barcodes will be compared. Cells will be modified with either 250 nm of 500 nm barcodes and then plated atop transwell membranes with 8 μm pores. Unlabeled MSC will be plated on the bottom of each transwell plate. If labeled MSC shed particles, cells on the bottom of the well plate will show evidence of labeling.

Barcode Self Destruction: The ability of the barcode to be destroyed during cell death significantly limits the possibility of barcodes being transferred to neighboring cells. To test the self-destructing characteristic of the barcode particles, cells will be labeled and then treated with compounds to induce apoptosis (staurosporine) or necrosis (ionomycin). Cells will be imaged hourly after exposure to chemical compounds to evaluate integrity of barcodes. If the barcode self-destructs, then the cell should have low levels of diffuse fluorescence rather than a punctate of bright fluorescence making it simple to assess if a cell is alive or in the process of dying. A subsample of cells will also be stained with the classic cell death marker propidium iodide to confirm barcode self-destruction occurred in dead cells.

Tracking Single Cells in a Multicellular Environment: To test the ability of barcodes to track cells over multiple generations in a multicellular environment, a scratch assay will be performed. MSCs will be grown to 70% confluence labeled with barcodes and then platted at high density. The next day, a scratch will be made in the monolayer using a pipette tip and cell migration to fill the void will be monitored with bright field and fluorescence microscopy. This assay will allow for tracking of cells over time and allow for examination of the relative contribution of cell division as well as the contribution of each cell to close the gap in the monolayer.

Identifying predictors of MSC in vivo survival: MSC ability to promote healing of damaged tissues make them applicable for dozens of conditions (13). However, almost all of the transplanted cells are known to die in a matter of days after transplantation, leaving behind only a small remnant of the initial population. It is not currently known if the surviving remnant is the result of random processes or if these cells have unique attributes that endow survival ability. To begin to analyze the question of why certain MSCs survive transplantation and demonstrate the utility of the proposed cell barcoding technique, MSC will be characterized in vitro for single cell secretion profile and then transplanted to assess cell survival. MSC secretion of TSG-6, TGF-beta, and VEGF will be measured using an ELISpot assay with custom-coated plates. MSCs will be plated and incubated for 24 hours on each plate. Before harvesting cells, the location of each cell will be documented and barcodes will be read. Plates will then be imaged, and the secretion of each cell will be determined by overlaying the coordinates of each cell with the presence of spots on the plate. The process will be repeated for each analyte. Cells will be detached from the plate using Cellstripper, a non-enzymatic reagent, to prevent damaging antibodies and bound analytes during harvesting.

MSC have been shown to home to sites of inflammation and injury (14). Therefore MSC survival will be assessed by measuring which MSC home and survive to a wound after intravenous delivery. Two dermal wounds will be created on the backs of NOD SCID mice using an 8 mm biopsy punch. 20,000 MSC will be injected into each animal via tail vein injection. After 7 days animals will be sacrificed and wounds will be harvested. One wound from each animal will be fixed and analyzed by confocal microscopy to identify MSC by their barcode while the other half will be enzymatically digested to produce a single cell suspension, plated, and barcodes read with a traditional fluorescence microscope. Thus, in vitro cell phenotype will be correlated to cell homing and 7 day persistence in a wound environment and determine if barcodes can be read in whole mount tissue.

### SUMMARY

The compositions and methods disclosed herein will significantly expand the questions biologists are capable of asking by enabling serial tracking of uniquely labeled individual cells over weeks and across multiple generations in a multicellular environment. This technology will allow a single cell to be phenotyped in multiple single cell assays to determine secretome, cell surface marker expression, motility characteristics, and then transferred to a multicellular environment where its interactions with other cells and host matrix can be further examined. The potential for this technology is enormous, especially when coupled with reporter gene technologies. By phenotyping cells prior to experiments in multicellular environments biomarkers will be determined that predict cell behavior. This invention can significantly impact the generation of cell-based therapies. For example, iPS derived insulin producing cells could be labeled as they are differentiated. Using this system, biomarkers that predict which cells will eventually differentiate into insulin producing cells can be identified days and weeks before differentiation occurs. Such knowledge would allow one to significantly enhance the efficiency of directed differentiation, which is currently limited by low yields. In addition, disclosed herein is the first self-destructing barcode designed to eliminate errors from transferred particles.

### Bibliography

1. Naik S H, Schumacher T N, Perie L. (2014). Cellular barcoding: a technical appraisal. Exp Hematol, 42(8), 598-608.
2. Gerrits A, Dykstra B, Kalmykowa O J, Klauke K, Verovskaya E, Broekhuis M J C, et al. (2010). Cellular barcoding tool for clonal analysis in the hematopoietic system. Blood, 115(13), 2610-8.
3. Yamanaka Y J, Szeto G L, Gierahn T M, Forcier T L, Benedict K F, Brefo M S N, et al. (2012). Cellular Barcodes for Efficiently Profiling Single-Cell Secretory Responses by Microengraving. Anal Chem, 84(24), 10531-6. PMCID: PMC3691955
4. Krutzik P O, Nolan G P. (2006). Fluorescent cell barcoding in flow cytometry allows high-throughput drug screening and signaling profiling. Nat Methods, 3(5), 361-8.
5. Mali P, Aach J, Lee J-H, Levner D, Nip L, Church G M. (2013). Barcoding cells using cell-surface programmable DNA-binding domains. Nat Methods Nature Publishing Group; 10(5), 403-6. PMCID: PMC3641172
6. Castellarnau M, Szeto G L, Su H W, Tokatlian T, Love J C, Irvine D J, et al. (2014). Stochastic Particle Barcoding for Single-Cell Tracking and Multiparametric Analysis. Small, n/a-n/a. 7. Rees P, Wills J W, Brown M R, Tonkin J, Holton M D, Hondow N, et al. (2014). Nanoparticle vesicle encoding for imaging and tracking cell populations. Nat Methods, 11(11), 1177-81.

8. Chithrani B D, Chan W C W. (2007). Elucidating the mechanism of cellular uptake and removal of protein-coated gold nanoparticles of different sizes and shapes. Nano Lett, 7(6), 1542-50.

9. Lee S K, Mortensen L J, Lin C P, Tung C-H. (2014). An authentic imaging probe to track cell fate from beginning to end. Nat Commun, 5,5216.

10. Ankrum J, Dastidar R G, Ong J F, Levy O, Karp J M. (2014). Performance-enhanced mesenchymal stem cells via intracellular delivery of steroids. Scientific Reports Nature Publishing Group; 4.
11. Ankrum J, Miranda O R, Ng K S, Sarkar D, Xu C, Karp J M. (2014). Engineering cells with intracellular agent-loaded microparticles to control cell phenotype. Nat Protoc, 9(2), 233-45.
12. Xu C, Miranda-Nieves D, Ankrum J, Matthiesen M E, Phillips J A, Roes I, et al. (2012). Tracking Mesenchymal Stem Cells with Iron Oxide Nanoparticle Loaded Poly(lactide-co-glycolide) Microparticles. Nano Lett [Internet], 12(8), 4131-9. Retrieved from: http://eutils.ncbi.nlm.nih.gov/entrez/eutils/elink.fcgi?dbfrom=pubmed&id=22769232&retmode=ref&cmd=prlinks. PMCID: PMC3552518
13. Ankrum J, Karp J. (2010). Mesenchymal stem cell therapy: Two steps forward, one step back. Trends Mol Med, 16(5), 203-9.
14. Karp J M, Leng Teo G S. (2009). Mesenchymal stem cell homing: the devil is in the details. Cell Stem Cell, 4(3), 206-16.

The invention is described with reference to various specific and preferred embodiments and techniques. However, it should be understood that many variations and modifications may be made while remaining within its scope. All referenced publications, patents and patent documents are intended to be incorporated by reference, as though individually incorporated by reference.

What is claimed is:

1. A barcode comprising a particle core of about 200 nm to 2000 nm in size and at least one fluorescent light emitting entity associated with said particle core.

2. The barcode of claim 1, wherein the core comprises a metal or polymer.

3. The barcode of claim 2, wherein the metal is gold, silver or a combination thereof.

4. The barcode of claim 2, wherein the polymer is PLGA, PLA, PGA, polystyrene, silicon, PCL or a combination thereof.

5. The barcode of claim 1, wherein the particle core is surface modified.

6. The barcode of claim 5, wherein the surface is modified so as to have biotin, streptavidin, an amino group, a carboxyl group, an antibody or combination thereof on the surface of the particle core.

7. The barcode of claim 1, wherein the at least one fluorescent entity is an organic fluorophore, a synthetic fluorophore, fluorescent nanocrystal, a polymeric fluorescing molecule or a combination thereof.

8. The barcode of claim 7, wherein the fluorescence entity is fluorescein, FITC, texas red, Alexa Fluor dyes, cyanine dyes or a combination thereof.

9. The barcode of claim 7, wherein the fluorescent nanocrystal is a quantum dot (QDot).

10. The barcode of claim 7, wherein the polymeric fluorescing molecule is a brilliant violet dye, a long-chain dialalkylcarbocyanine dye, or a combination thereof.

11. The barcode of claim 1, wherein the at least one fluorescent entity is directly or indirectly attached to the core.

12. The barcode of claim 11, wherein the at least one fluorescent entity is attached to the core by an antibody, a biotin/streptavidin attachment, electrostatic interactions or a combination thereof.

13. The barcode of claim 12, wherein multiple antibodies with fluorophores are stacked on the core.

14. The barcode of claim 12, wherein there are sequential biotin-streptavidin interactions.

15. The barcode of claim 12, wherein there are multiple layers of fluorophores held by electrostatic interactions.

16. The barcode of claim 15, wherein the electrostatic interactions are created by alternating layers of PLL and PAA.

17. The barcode of claim 1, further comprising poly-D-lysine.

18. A method to label a cell comprising contacting said cell with said barcode of claim 1.

19. A method to identify and/or track cells comprising measuring the fluorescent intensity from the barcode in the labeled cell of claim 18.

20. The method of claim 19, wherein the barcode is read at more than one time point.

21. The method of claim 18, wherein the barcode is passed to multiple cell generations.

22. The method of claim 18, wherein the barcode is stable for two or more weeks.

23. The method of claim 18, wherein the barcode degrades upon cell death.

24. The method of claim 20, wherein the barcode can be detected by flow cytometry, fluorescent microscopy, high content imaging or a combination thereof.
